(19) United States
(12) Patent Application Publication
(10) Pub. No.: US 2020/0215198 A1
(43) Pub. Date: Jul. 9, 2020

(54) COMPOSITIONS AND REAGENTS FOR ION BEAM IMAGING

(71) Applicant: IONpath, Inc., Menlo Park, CA (US)

(72) Inventors: Sean Bendall, San Mateo, CA (US); Jason Ptacek, Menlo Park, CA (US)

(21) Appl. No.: 16/733,360

(22) Filed: Jan. 3, 2020

Related U.S. Application Data
(60) Provisional application No. 62/788,118, filed on Jan. 3, 2019.

(51) Int. Cl. A61K 47/60; A61K 51/08; C07K 17/14

(52) U.S. Cl. CPC A61K 47/60; C07K 17/14; A61K 51/088

(57) ABSTRACT

Compositions include a labeling agent featuring a plurality of free labeling units, where each labeling unit includes at least one conjugating group for covalent bonding to a biological molecule, at least one metal chelating group, and a metal ion chelated by the at least one metal chelating group, where a percentage by weight of water in the composition is 10% or less.

### CROSS-REFERENCE TO RELATED APPLICATIONS

This application claims priority to U.S. Provisional Patent Application No. 62/788,118, filed on Jan. 3, 2019, the entire contents of which are incorporated herein by reference.

### TECHNICAL FIELD

This disclosure relates to scanning of biological samples using an ion beam, and to preparation of samples for scanning.

### BACKGROUND

Immunohistochemistry methods have been used to visualize protein expression in biological samples such as tumor tissue biopsies. Such methods typically involve exposing a sample to antibodies coupled to fluorescent moieties or enzyme reporters that generate colored pigments. Analysis of spectral images of the tagged sample yields information that can be used to assess protein expression levels and co-expression events. A variety of samples can be analyzed using such methods, including formalin-fixed, paraffin-embedded tissue sections.

### SUMMARY

Ion beam scanning and imaging of biological samples is a powerful technique for providing insight into functional relationships between protein expression, nucleic acids, and other structural components of samples. Information about co-regulation and co-expression within individual cells and cellular compartments can be used to elucidate mechanisms of disease and evaluate pharmaceuticals and other therapeutic methods.

Certain ion beam imaging methods involve exposing a sample to a beam of primary ions from an ion source. The primary ions generate secondary ions from the sample which are detected. In general, specific types of secondary ions are correlated with specific analytes within a sample so that each type of analyte can be selectively and quantitatively measured. In some methods, the secondary ions that are generated are mass tags, with a specific mass-to-charge ratio.

Prior to exposing the sample to the primary ion beam, the sample can be prepared with suitable mass tags. In general, suitable preparation methods involve binding specific mass tags to specific analytes (e.g., bio-molecules and/or structural moieties) within the sample. When secondary ions corresponding to the mass tags are generated, individual types of secondary ions (with specific mass-to-charge ratios) are attributed to the specific analytes to which the corresponding mass tags were bound.

This disclosure features reagents for sample preparation prior to ion beam imaging, and methods for preparing samples using the reagents. In general, the reagents include a labeling agent that includes one or more binding sides for mass tags, and mass tags bound to at least some of the one or more binding sites. A variety of different mass tags can be used. In some embodiments, the mass tags can include metal atoms or ions, and in particular, lanthanide metal atoms or ions. By using mass tags that correspond to metal atoms or ions, the secondary ions generated from the sample are metal ions with specific mass-to-charge ratios. Such secondary ions are readily detected and discriminated.

In a first aspect, the disclosure features compositions that include a labeling agent featuring a plurality of free labeling units, where each labeling unit includes at least one conjugating group for covalent bonding to a biological molecule, at least one metal chelating group, and a metal ion chelated by the at least one metal chelating group, where a percentage by weight of water in the composition is 10% or less.

Embodiments of the compositions can include any one or more of the following features.

Each free labeling unit can be an oligomeric unit formed from multiple conjugated monomer units. Each oligomeric unit can include at least n conjugated monomer units, e.g., where n is 3 or more, or 5 or more. Each oligomeric unit can include the same number of conjugated monomer units.

At least one of the free oligomeric units can include a first number of conjugated monomer units, and at least one of the free oligomeric units can include a second number of conjugated monomer units that is different from the first number of conjugated monomer units. At least one of the free oligomeric units can include one or more first monomer units and one or more second monomer units different from the first monomer units. A ratio of the first monomer units to the second monomer units in the at least one of the free oligomeric units can be between 50:1 and 1:50.

The first and second monomer units can be alternately conjugated in the at least one of the free oligomeric units. At least two of the first monomer units can be conjugated to one another in the at least one of the free oligomeric units. At least one of the free oligomeric units can include one or more first monomer units and one or more third monomer units different from the first and second monomer units.

The monomer units can include one or more amino acids. The one or more amino acids can include at least one member selected from the group consisting of lysine, serine, arginine, glutamine, asparagine, histidine, threonine, tyrosine, cysteine, alanine, isoleucine, leucine, methionine, phenylalanine, valine, proline, and glycine.

One or more of the oligomeric units can include a peptide. The monomer units can include one or more organic monomers. The one or more organic monomers can include at least one member selected from the group consisting of methacrylates and their derivatives, vinyls and their derivatives, ethylenes and their derivatives, propylenes and their derivatives, sulfones and their derivatives, and urethanes and their derivatives.

At least one oligomeric unit can include multiple monomer units conjugated to form a linear structural moiety. At least one oligomeric unit can include multiple monomer units conjugated to form a dendrimer. Each free labeling unit can be a nanoparticle.

The at least one conjugating group can include at least one member selected from the group consisting of: a succinimidyl 4-(N-maleimidomethyl)cycicohexane-1-carboxylate) group, an N-hydroxysuccinimide group, a maleimide group, and an isothiocyanate group.

The at least one conjugating group can include a moiety that reacts with an amine group of a biological molecule. The at least one conjugating group can include a moiety that reacts with a sulfhydryl group of a biological molecule. The at least one conjugating group can include a moiety that reacts with a carboxylic acid group of a biological molecule. The at least one conjugating group can include a moiety that reacts with acetylene. The at least one conjugating group can include a moiety that reacts with an azide group or ion.

At least one of the free labeling units can include multiple conjugating groups (e.g., two or more conjugating groups, three or more conjugating groups). At least one of the multiple conjugating groups can be different from another one of the multiple conjugating groups.

At least one free labeling unit can include multiple metal chelating groups (e.g., three or more metal chelating groups, five or more metal chelating groups). The at least one free labeling unit can include at least one first metal chelating group and at least one second metal chelating group different from the first metal chelating group.

The at least one metal chelating group can include at least one member selected from the group consisting of: a 1,4,7,10-tetraazacyclododecane-1,4,7,10-tetraacetic acid group and conjugates thereof, a diethylenetriaminepentaacetic acid group and conjugates thereof, an ethylenediaminetetraacetic acid group and conjugates thereof, and a 1,4,7-tricarboxymethyl-1,4,7-triazacyclononane group and derivatives thereof.

The at least one metal chelating group can include at least one group having two chelating moieties. The at least one metal chelating group can include at least one group having three chelating moieties. The at least one metal chelating group can include at least one group having four chelating moieties. At least one free labeling unit can include a first metal chelating group featuring n chelating moieties, and a second metal chelating group different from the first metal chelating group and featuring m chelating moieties, where n and m are different.

At least one free labeling unit can include a first metal ion and a second metal ion different from the first metal ion. The first and second metal ions can have different atomic numbers. The first and second metal ions can be different isotopes and can have a common atomic number. A concentration ratio of the first metal ion to the second metal ion can be 95:5 or more. At least one free labeling unit can include two or more metal ions (e.g., three or more metal ions, five or more metal ions).

The composition can include a plurality of anions such that the composition forms a salt. The plurality of anions can include at least one member of the group consisting of chloride ions, fluoride ions, iodide ions, bromide ions, nitrate ions, acetate ions, and oxide ions.

Embodiments of the compositions can also include any of the other features disclosed herein, including any combinations of features disclosed in connection with different embodiments, except as expressly stated otherwise.

In another aspect, the disclosure features methods that include providing a labeling agent featuring a plurality of free labeling units, where each labeling unit includes at least one conjugating group for covalent bonding to biological molecule, at least one metal chelating group, and a metal ion chelated by the at least one metal chelating group, where a percentage by weight of water in the labeling agent is 10% or less, conjugating the labeling agent to a biological molecule to form a conjugated labeling reagent, and stabilizing the conjugated labeling reagent.

Embodiments of the methods can include any one or more of the following features.

The stabilized conjugated labeling reagent can have a storage lifetime of at least two weeks at room temperature.

Stabilizing the conjugated labeling reagent can include adjusting a percentage by weight of water in the conjugated labeling reagent to 10% or less. The methods can include extracting water from the conjugated labeling reagent by at least one of dehydrating and centrifuging the conjugated labeling reagent. The methods can include extracting water from the conjugated labeling reagent by combining the conjugated labeling reagent with an organic solvent.

Stabilizing the conjugated labeling reagent can include reducing a temperature of the conjugated labeling reagent to −20 degrees Celsius or less, and maintaining the reduced temperature. Stabilizing the conjugated labeling reagent can include at least one of adjusting and maintaining a pH of the conjugated labeling reagent to or at a value of 6.5 or less (e.g., a value of 6 or less).

Stabilizing the conjugated labeling reagent can include combining the conjugated labeling reagent with a preservation agent. The preservation agent can include at least one member selected from the group consisting of a carbohydrate and a polymer. The preservation agent can include at least one member selected from the group consisting of trehalose, maltose, sucrose, polyethylene glycol, and gelatin.

Stabilizing the conjugated labeling reagent can include treating the at least one conjugating group with a protecting agent to covalently bind a protecting group to the at least one conjugating group. The protecting group can include at least one member selected from the group consisting of dimethylfuran and its derivatives and isomers such as 2,5-dimethylfuran, methylfuran and its derivatives and isomers such as 2-methylfuran, and chemical groups that form alkoxymethyl ethers such as methoxymethyl ethers and ethoxymethyl ethers.

Embodiments of the methods can also include any of the other features disclosed herein, including any combinations of features disclosed in connection with different embodiments, and any combinations of features disclosed in connection with the compositions, except as expressly stated otherwise.

In a further aspect, the disclosure features reagent kits that include a labeling agent featuring a plurality of free labeling units, where each labeling unit includes at least one conjugating group for covalent bonding to a biological molecule, at least one metal chelating group, and a metal ion chelated by the at least one metal chelating group, and further including at least one member of the group consisting of instructions for preparing a conjugate of the labeling agent, one or more buffer solutions, one or more purification devices, and a stabilization solution for storage of a conjugate of the labeling agent.

Embodiments of the reagent kits can include any one or more of the following features.

The one or more purification devices can include at least one of a MWCO column and a gel permeation chromatography column.

Embodiments of the reagent kits can also include any of the other features disclosed herein, including any combinations of features disclosed in connection with different embodiments, and any combinations of features disclosed in connection with the compositions, except as expressly stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1 is a schematic diagram showing an example of a labeling unit for use in preparing samples for multiplexed ion beam imaging.

FIGS. 2A-2F are schematic diagrams showing examples of structural backbones.

FIG. 3 is a flow chart showing a series of example steps for binding metal ions to metal chelating groups.

FIG. 4 is a flow chart showing a series of example steps for producing a conjugated labeling reagent by covalently bonding conjugating groups to antibodies.

FIG. 5 is a schematic diagram showing a sample on a substrate.

FIG. 6 is a schematic diagram showing a sample on a substrate that includes multiple sample wells.

FIG. 7 is a schematic diagram showing an example system for multiplexed ion beam imaging of sample.

FIG. 8 is a graph showing the amount of antibody recovered following conjugation of free labeling units to form a conjugated labeling reagent.

FIG. 9 is a graph showing abundance of mass tags per antibody molecule for antibodies conjugated with freshly prepared labeling compositions and labeling compositions that have been prepared previously and stored.

FIG. 10 shows multiplexed ion beam images of colon tissue.

FIG. 11 shows multiplexed ion beam images of placenta tissue.

FIG. 12 is a graph showing relative metal tag loading concentration levels for different labeling reagents.

Like reference symbols in the various drawings indicate like elements.

### DETAILED DESCRIPTION

### (i) Introduction

Multiplexed visualization of protein expression and other biochemical moieties and structures allows researchers to identify important correlations between biological functional events. Visualization of protein expression can be used to assess malignancies in excised tissue samples as part of a diagnostic work-up, and in particular, to provide important information about signaling pathways and correlated structural development in tumor tissue.

Conventional multiplexed immunohistochemical techniques for visualizing protein expression typically rely on optical detection of fluorescence emission from a sample that has been labeled with multiple antibody-conjugated flurophores. The conjugated fluorophores bind specifically to corresponding antigens in the sample, and imaging of fluorescence emission from the sample is used to assess the spatial distribution of the fluorophores. For samples in which antigen concentrations are relatively low, signal amplification (e.g., using multivalent, enzyme-linked secondary antibodies) can be used to aid visualization. However, the use of signal amplification techniques can compromise quantitative information (e.g., antigen concentration information) that might otherwise be extracted from sample images.

In conventional multiplexed immunohistochemical visualization techniques, other constraints can also be encountered. Optical detection and separation of spectral signatures of multiple fluorophores is a complex problem, particularly where the fluorescence spectra of the fluorophores exhibit significant overlap. Without robust discrimination between spectral signatures of the fluorophores, important expression-related information is not uncovered. Further, such techniques often rely on primary antibodies generated in dissimilar host species. These factors can limit the utility of conventional multiplexed immunohistochemical visualization techniques for predictive biomarker development and clinical diagnostics.

Secondary ion mass spectrometry can be used as an alternative technique for performing multiplexed visualization of antigens and other biochemical structures and moieties in biological samples. Structure-specific antibodies are conjugated to specific mass tags, typically in the form of metallic elements (e.g., lanthanide elements). When a sample is exposed to a labeling agent that includes a mass tag and a binding group, the labeling agent binds to a complementary moiety in the sample. As one example, labeling agents can include antibodies that specifically bind to corresponding antigens in the sample.

Exposure of the labeled sample to a primary ion beam liberates secondary ions corresponding to the conjugated mass tags from the labeled sample. Performing spatially-resolved detection of the secondary ions that are generated from the sample allows direct visualization of the localization of specific antigens in the sample, and extraction of quantitative information (e.g., antigen concentration) as a function of spatial location. This information can be combined with other structural information (e.g., information about tumor margins, cell types/morphologies) to develop a detailed assessment of tumor viability and progression in the sample.

The methods disclosed herein, which are referred to as multiplexed ion beam imaging (MIBI) methods, can be used to resolve spatial distributions of relatively large numbers of mass tags applied to samples. For example, visual and quantitative assessment of up to 100 different mass tags in a single sample are possible. Depending upon the nature of the mass tags applied to the sample, sensitivities in the parts-per-billion range can be achieved with a dynamic range of approximately 10⁵. Imaging resolution is typically comparable to optical microscopy at high magnification.

Conventional methods of sample preparation to introduce mass tags suitable for use in MIBI methods can be challenging to implement for a variety of reasons. In general, such methods involve a sequence of controlled steps that are carefully performed under relatively restrictive pH, temperature, and other conditions. The overall preparation time can be lengthy—on the order of six hours or more—resulting in a relatively extended overall measurement process. Further, the complexity of such preparative procedures can lower performance, particularly when the preparation is carried out by users who are not highly experienced.

In addition, certain types of mass tags—while they are well-suited for detection as secondary ions—can have deleterious effects on samples. In particular, certain heavy metal elements can cause chemical and/or physical changes in sample components (such as CsCl precipitation of DNA) even when the elements are present in solution at relatively low concentration. Preventing certain types of mass tags from causing unacceptable sample modification can be difficult.

Further still, conventional methods of sample preparation typically involve an in situ loading step in which a multimeric chelator is “loaded” with mass tags (e.g., metal atoms). Because certain metal atoms are relatively insoluble in solutions at pH values of approximately 7, introducing such metal atoms may involve reducing the solution pH to a value that is significantly more acidic. However, at relatively acidic pH values, bio-reactive functional groups (such as antibodies) can be hydrolyzed during chelation and washing steps, such that the overall yield of the sample labeling process is reduced.

In contrast to methods that involve in situ preparation of a labeling agent and then exposure of the labeling agent to the sample, the methods discussed herein involve preparation and isolation of a labeling agent as an intermediate. The isolated labeling agent includes at least one conjugating group for covalent bonding to a biological molecule, at least one metal chelating group, and at least one metal atom or ion (such as a lanthanide metal atom or ion) bound to the chelating group. The labeling agent is stabilized as part of the isolation process so that the metal ion mass tag(s) remain bound to the chelating group(s). A sample-binding moiety (such as an antibody or a nucleic acid fragment) is then conjugated to the isolated labeling agent to form a sample label, and the sample is exposed to the sample label to complete preparation of the sample.

Preparing samples in the foregoing manner can overcome some of the complexities associated with conventional sample preparative methods. Overall sample preparation time can be reduced, and the likelihood that the preparative steps will interfere with sample binding groups or result in undesirable sample modification can be reduced. The intermediate labeling agents can be prepared well in advance and stored for a period of one year or more prior to use, without significant apparent adverse consequences.

### (ii) Labeling Units

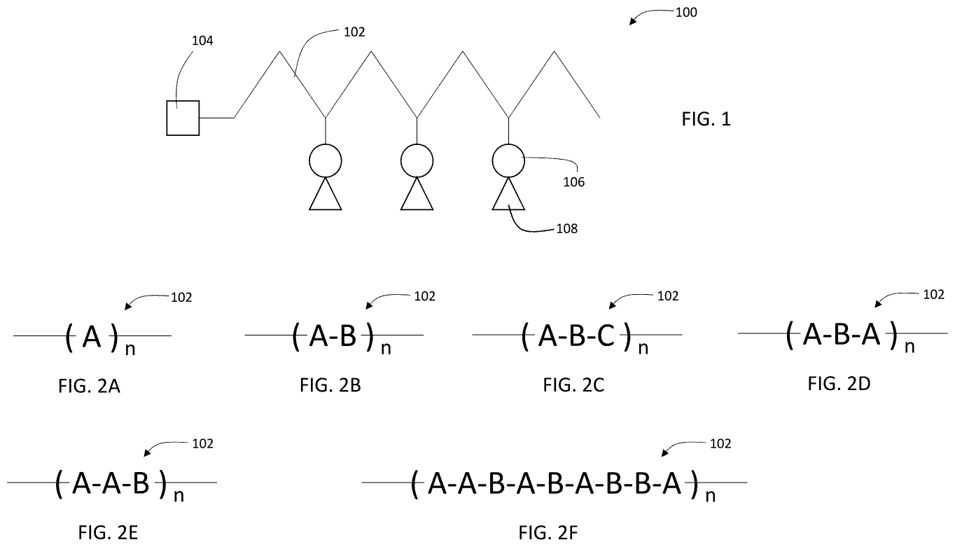

FIG. 1 is a schematic diagram showing an example of a labeling unit 100 for use in preparing samples for MIBI imaging. Labeling unit 100 includes a structural backbone 102, at least one conjugating group 104, at least one metal chelating group 106, and at least one metal atom or ion 108 chelated to the metal chelating group(s) 106.

In general, the labeling agent compositions described herein include a plurality of free labeling units 100. That is, such compositions typically include at least some labeling units 100 that are not bound to a sample or to each other. The free labeling units 100 form a labeling agent which can then be further prepared for direct exposure to the sample, as will be discussed in greater detail below.

A wide variety of different structural backbones can be used in labeling unit 100. In some embodiments, for example, structural backbone 102 is an oligomeric moiety formed from multiple conjugated monomer units. For example, structural backbone 102 can be formed from at least n conjugated monomer units, where n is 2 or more (e.g., 3 or more, 4 or more, 5 or more, 6 or more, 8 or more, 10 or more, 15 or more, 20 or more, or even more).

In certain embodiments, each free labeling unit 100 in a labeling agent has a structural backbone 102 that is formed from the same number of conjugated monomer units. Alternatively, in some embodiments, a labeling agent can include free labeling units 100 with structural backbones 102 formed from different numbers of conjugated monomer units. For example, a labeling agent can include one or more free labeling units 100 with a structural backbone 102 formed from j conjugated monomer units, and one or more free labeling units 100 with a structural backbone 102 formed from k conjugated monomer units, where j and k are different. Further still, the labeling agents described can include free labeling units 100 with structural backbones 102 formed from more than two different numbers of conjugated monomer units. That is, in general, the labeling agents can include free labeling units 100 with structural backbones formed from two or more (e.g., three or more, four or more, five or more, six or more, eight or more, ten or more, or even more) different numbers of conjugated monomer units.

In some embodiments, structural backbone 102 can be formed of multiple conjugated monomer units, where each monomer unit is the same. That is, structural backbone 102 can correspond to a monoblock oligomer. FIG. 2A shows a schematic diagram of a monoblock oligomer structural backbone 102, consisting of only one type of monomer unit A. Alternatively, in certain embodiments, structural backbone 102 can be formed of different types of conjugated monomer units. For example, FIG. 2B shows a schematic diagram of a structural backbone 102 formed of both A and B monomer units, as a diblock oligomer. FIG. 2C shows a schematic diagram of a structural backbone 102 formed of A, B, and C monomer units, as a triblock oligomer. More generally, structural backbone 102 can be formed of any number of different types of conjugated monomer units, e.g., 2 or more (3 or more, 4 or more, 5 or more, 6 or more, 8 or more, 10 or more, or even more) different types of conjugated monomer units.

In certain embodiments, as shown in FIGS. 2B and 2C, where structural backbone 102 includes different types of monomer units, the monomer units are present in equimolar ratios in structural backbone 102. Alternatively, in some embodiments, different types of monomer units can be present in non-equimolar ratios. For example, FIGS. 2D and 2E show examples of structural backbones 102 in which the ratio of A monomer units to B monomer units is 2:1. More generally, monomer units in structural backbone 102 can be present is any ratio as desired to yield a free labeling unit 100 with suitable properties (e.g., solubility, binding affinity, conformational shape). As another example, two different monomer units in a structural backbone 102 of a free labeling unit 100 can be present in a ratio of between 50:1 and 1:50 (e.g., between 20:1 and 1:20, between 10:1 and 1:10, between 1:5 and 5:1).

In certain labeling agents, the labeling units 100 each have the same structural backbone 102 and other features shown in FIG. 1, and thus, the dispersity index of such labeling agents is 1. Alternatively, in some labeling agents, different labeling units can have structural backbones 102 of different lengths and/or different compositions. For such labeling agents, the dispersity index can be 1.01 or more (e.g., 1.05 or more, 1.10 or more, 1.20 or more, 1.30 or more, 1.50 or more, 1.70 or more, 2.0 or more, 3.0 or more, or even more).

In the structural backbones 102 shown in FIGS. 2B-2E, monomer units are regularly conjugated to form a repeating block within the structural backbone. In some embodiments, however, at least a portion of structural backbone 102, or the entire structural backbone, can be formed from two or more different types of monomer units that are conjugated irregularly, such that the structural backbone is not formed (or not entirely formed) from a repeating block. An example of such a structural backbone 102 is shown in FIG. 2F.

Structural backbone 102 can be formed from a wide variety of different conjugated monomers. Examples of individual monomers are discussed below. However, it should be understood that, as described above, combinations of any two or more different types of monomers can form structural backbone 102, provided that a synthetic route to conjugation of the different types of monomers is available.

In some embodiments, the monomer units that are conjugated to form structural backbone 102 include one or more amino acids. Suitable amino acids include, for example, lysine, serine, arginine, glutamine, aspartic acid, glutamic acid, asparagine, histidine, threonine, tyrosine, cysteine, tryptophan, alanine, isoleucine, leucine, methionine, phenylalanine, valine, proline, glycine, and structural derivatives of these amino acids. Both L- and D-forms of the foregoing amino acids and derivatives (with the exception of glycine, which has no enantiomers) can be used. When structural backbone 102 is formed from combinations of one or more amino acids, structural backbone 102 can correspond to a peptide.

In certain embodiments, the monomer units that are conjugated to form structural backbone 102 include one or more nucleic acid fragments. Suitable nucleic acid fragments include single-stranded DNA fragments, and a variety of different types of RNA fragments such as, but not limited to, pRNA-3WJ and box C/D RNA building blocks. Suitable nucleic acid fragments are disclosed for example in Li et al., “RNA as a stable polymer to build controllable and defined nanostructures for material and biomedical applications,” Nano Today 10(5): 631-655 (2015), the entire contents of which are incorporated herein by reference. When structural backbone 102 is formed from combinations of nucleic acid fragments, structural backbone 102 can correspond to a poly-nucleic acid moiety.

In some embodiments, the monomer units that are conjugated to form structural backbone 102 include one or more monosaccharides. Suitable monosaccharides include, for example, glucose, fructose, galactose, mannose, ribose, and deoxyribose, and structural derivatives of monosaccharides. Both L- and D-forms of the foregoing monosaccharides and derivatives can be used. Alternatively, or in addition, the monomer units that are conjugated to form structural backbone 102 can include one or more disaccharides. Suitable disaccharides include, for example, sucrose, lactose, maltose, and derivatives thereof. When structural backbone 102 is formed from combinations of monosaccharides and/or disaccharides, structural backbone 102 can correspond to a polysaccharide or, more generally, a carbohydrate moiety.

In certain embodiments, the monomer units that are conjugated to form structural backbone 102 include one or more organic monomers. Generally, a wide variety of different monomers can be used. Examples of suitable monomers include, but are not limited to, methacrylates (e.g., methyl methacrylate) and their derivatives, vinyls (e.g., vinyl chloride) and their derivatives, ethylenes and their derivatives, propylenes and their derivatives such as poly(trimethylene-1,1-dicarboxylate), sulfones and their derivatives, and urethanes and their derivatives.

In some embodiments, structural backbone 102 can include a moiety that is not formed from conjugated monomers. For example, structural backbone 102 can be a lipid. Examples of suitable lipids include, but are not limited to, glycerol and sphingosine lipid derivatives. Further examples of suitable structural backbones 102 that are not formed from conjugated monomers include, but are not limited to, carbohydrate-based backbones and amino acid-based backbones.

In certain embodiments, structural backbone 102 can be a nanoparticle. Aspects and features of nanoparticles that can be used as structural backbone 102 are disclosed, for example, in Liu et al., “Metal chelators coupled with nanoparticles as potential therapeutic agents for Alzheimer's disease,” J. Nanoneurosci. 1(1): 42-55 (2009), and in Bonvin et al., “Chelating agents as coating molecules for iron oxide nanoparticles,” RSC Adv. 7: 55598-55609 (2017). The entire contents of each of the foregoing references are incorporated by reference herein.

The examples of structural backbone 102 shown in FIGS. 2A-2F are linear. That is, in each of these figures, structural backbone 102 is formed from multiple monomer units conjugated to form a linear structural moiety. In general, a wide variety of different linear structural backbones 102 can be used, including both branched and unbranched linear structural moieties.

Alternatively, in certain embodiments, structural backbone 102 can be a dendrimeric structural moiety. Suitable dendrimeric structural moieties include, but are not limited to, dendrimers, dendrons, and various polydisperse dendrimeric hybrid structures such as hyperbranched structures, dendrigrafts, and dendritic-linear hybrid structural moieties.

Returning to FIG. 1, a variety of different conjugating groups 104 can be used in free labeling unit 100. In general, conjugating group 104 facilitates attachment of structural backbone 102 to a biological molecule which, in turn, binds to a molecular entity (such as a bio-molecule or structural moiety) within a sample. As such, a variety of different conjugating groups 104 can be used, depending upon the nature of the biological molecule that will be covalently bound to structural backbone 102. Suitable conjugating groups include, but are not limited to, succinimidyl 4-(N-maleimidomethyl)cyclohexane-1-carboxylate) groups and their derivatives, N-hydroxysuccinimide groups and their derivatives, maleimide groups and their derivatives, isothiocyanate groups and their derivatives, and amine-reactive chemical groups such as, but not limited to, N-hydroxysuccinimide esters and imidoesters.

In certain embodiments, conjugating group 104 is a functional group or structural moiety that reacts with a target group on a biological molecule via a “click chemistry” reaction. In general terms, click chemistry reactions suitable for implementation include a variety of thermodynamically favored reactions, including nucleophilic ring opening reactions of epoxides and aziridines, non-aldol type carbonyl reactions (e.g., formation of hydrazones and heterocycles), additions to carbon-carbon multiple bonds (e.g., oxidative formation of epoxides and Michael additions), and cycloaddition reactions. Azide-alkyne cycloadditions, which can be catalyzed by metals such as copper and ruthenium complexes, are one example of suitable click chemistry reactions. Additional examples of click chemistry reactions and associated conjugating groups are described in U.S. Pat. No. 6,800,728, and in Horisawa, “Specific and quantitative labeling of biomolecules using click chemistry,” Front. Physiol. 5: 457 (2014). The entire contents of each of the foregoing references are incorporated herein by reference.

More generally, conjugating group 104 can be any functional group or structural moiety that reacts with a target group on a biological molecule, to yield a covalent bond between the biological molecule and free labeling unit 100. Accordingly, suitable conjugating groups 104 include, but are not limited to, moieties that react with an amine group of a biological molecule, moieties that react with a sulfhydryl group of a biological molecule, moieties that react with a carboxylic acid group of a biological molecule, moieties that react with acetylene, and moieties that react with an azide group or ion.

In FIG. 1, free labeling unit 100 includes a single conjugating group 104. More generally, however, free labeling units can include one or more (e.g., two or more, three or more, four or more, five or more, or even more) conjugating groups 104. When free labeling unit 100 includes more than one conjugating group 104, each of the conjugating groups 104 can be the same, or some of the conjugating groups may differ from one another. Using multiple conjugating groups 104 that are the same may facilitate binding of the free labeling unit to a biological molecule, as multiple covalent binding locations are available for attachment of the biological molecule.

Alternatively, using different conjugating groups 104 allows different biological molecules to be bound to free labeling unit 100. For example, different conjugating groups may allow different antibodies, or different nucleic acids, or a combination of antibodies and nucleic acids, to be bound to free labeling unit 100. This flexibility allows for synthesis of a free labeling unit 100 that is capable of binding to more than one type of molecular entity within a sample, such that the free labeling unit can be used in different labeling compositions to target different types of analytes.

Returning again to FIG. 1, free labeling unit 100 can generally include one or more (e.g., two or more, three or more, four or more, five or more, six or more, eight or more, ten or more, or even more) metal chelating groups 106. In some embodiments, each of the metal chelating groups in free labeling unit 100 are the same. Alternatively, in certain embodiments, at least some of the metal chelating groups 106 differ from one another. By using different metal chelating groups, it can be possible to bind different types of metal atoms or ions to the same free labeling unit 100. Further, binding the same type of metal atoms or ions to different types of chelating groups in a free labeling unit 100 can allow for selective generation of secondary ions at different primary ion energies, depending upon the relative strengths of the coordination between the different types of chelating groups and the bound metal atoms or ions.

Generally, a wide variety of different chelating groups 106 can be used in free labeling unit 100. Examples of such groups include, but are not limited to, 1,4,7,10-tetraazacyclododecane-1,4,7,10-tetraacetic acid groups and derivatives thereof, diethylenetriaminepentaacetic acid groups and derivatives thereof, ethyl enediaminetetraacetic acid groups and derivatives thereof, and 1,4,7-tricarboxymethyl-1,4,7-triazacyclononane groups and derivatives thereof.

In some embodiments, one or more of the metal chelating groups 106 includes two or more chelating moieties. Typically, metal chelating groups with multiple chelating moieties can be used to chelate metal ions that carry multiple positive charges (e.g., +2, +3, +4). Accordingly, in some embodiments, free labeling unit 100 includes at least one metal chelating group 106 with two or more (e.g., three or more, four or more, five or more, or even more) metal chelating moieties.

In certain embodiments, where free labeling unit 100 includes different metal chelating groups 106, the different metal chelating groups can have the same number, or different numbers, of chelating moieties. For example, free labeling units 100 can include at least one chelating group 106 of a first type, with g chelating moieties per chelating group, and at least one chelating group 106 of a second type, with h chelating moieties per chelating group, where g and h can be the same or different.

Free labeling unit 100 also includes one or more metal atoms or ions 108 bound to at least one or more of metal chelating groups 106. While in FIG. 1 each of the metal chelating groups 106 is bound to a corresponding metal atom or ion 108, more generally, some of the metal chelating groups 106 may not be bound to corresponding metal atoms or ions in free labeling unit 100. Typically, free labeling unit 100 can include, at most, as many metal atoms or ions 108 as there are metal chelating groups 106. Accordingly, an individual free labeling unit 100 can include one or more (e.g., two or more, three or more, four or more, five or more, six or more, eight or more, ten or more, 15 or more, 20 or more, or even more) metal atoms or ions 108.

Metal atoms or ions 108 can generally include various metals selected as desired to yield secondary ions that can be quantitatively detected and correlated with the presence of specific analytes in the sample. In some embodiments, metal atoms or ions 108 include lanthanide metal ions (e.g., lanthanum, neodymium, samarium, gadolinium, erbium, ytterbium, and/or dysprosium ions). Alternatively, or in addition, in certain embodiments, metal atoms or ions 108 include other heavy metals such as bismuth, yttrium, indium, gallium, zirconium, ruthenium, and chromium. Such metals—which may be relatively insoluble in a variety of typical preparative solutions—are nonetheless particularly amenable to inclusion in free labeling unit 100, as they can be used to label a sample according to a preparative scheme that reduces precipitation of salts of such metals from solution, compared with conventional sample preparative methods.

In certain embodiments, free labeling unit 100 can include multiple different types of metal atoms or ions 108. For example, an individual free labeling unit 100 can include two or more (e.g., three or more, four or more, five or more, or even more) different types of metal atoms or ions. In some embodiments, the different types of metal atoms or ions have different atomic numbers, and therefore correspond to different metallic elements.

Alternatively, in certain embodiments, the different types of metal atoms or ions have the same atomic number but different atomic masses, and therefore correspond to different isotopes of the same metallic element. Where free labeling unit 100 includes atoms or ions 108 corresponding to two different isotopes, for example, a concentration ratio of the first isotope to the second isotope in a composition that includes a plurality of free labeling units 100 can be 95:5 or more (e.g., 97:3 or more, 98:2 or more, 99:1 or more, 99.5:0.5 or more).

As described above, free labeling agent 100 can generally include a wide variety of different structural backbones 102, conjugating groups 104, metal chelating groups 106, and metal atoms or ions 108. As a result, a significantly wide range of different synthetic schemes can be used to prepare the different types of free labeling agents described.

Examples of free labeling agents 100 based on linear and branched linear structural backbones are disclosed in: Lou et al., “Polymer-based elemental tags for sensitive bioassays,” Angew. Chem. Int. Ed. Engl. 46(32): 6111-6114 (2007); Majonis et al., “Synthesis of a functional metal-chelating polymer and steps toward quantitative mass cytometry bioassays,” Anal. Chem. 82(21): 8961-8969 (2010); Majonis et al., “Curious results with palladium- and platinum-carrying polymers in mass cytometry bioassays and an unexpected application as a dead cell stain,” Biomacromolecules 12(11): 3997-4010 (2011); and Illy et al., “Metal-chelating polymers by anionic ring-opening polymerization and their use in quantitative mass cytometry,” Biomacromolecules 13): 2359-2369 (2012). Examples of free labeling agents 100 based on branched and/or dendritic structural backbones 102 are disclosed in: Xu et al., “Preparation and preliminary evaluation of a biotin-targeted, lectin-targeted, dendrimer-based probe for dual-modality magnetic resonance and fluorescence imaging,” Bioconjug. Chem. 18(5): 1474-1482 (2007); Rahmania et al., “Synthesis and stability test of radiogadolinium(III)-DOTA-PAMAM G3.0-trastuzumab as SPECT-MRI molecular imaging agent for diagnosis of HER-2 positive breast cancer,” J. Rad. Research Appl. Sciences 8(1): 91-99 (2015); Nwe et al., “Preparation of cystamine core dendrimer and antibody-dendrimer conjugates for MRI angiography,” Mol. Pharm. 9(3): 374-381 (2012); PCT Patent Application Publication No. WO 95/24221, entitled “Bioactive and/or targeted dendrimer conjugates”; and EP Patent No. 0271180, entitled “Starburst Conjugates”. The entire contents of each of the foregoing references are incorporated by reference herein.

(iii) Compositions of Labeling Agents

After structural backbone 102 has been prepared with one or more conjugating groups 104 and one or more metal chelating groups 106, preparation of free labeling agent 100 is completed by binding metal atoms or ions 108 to at least one of the metal chelating groups 106. In general, binding metal atoms or ions 108 to metal chelating groups 106 is performed by exposing the metal chelating groups 106 to the metal atoms or ions 108 in solution.

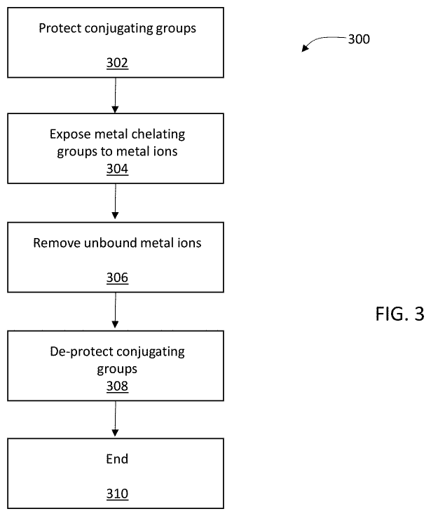

FIG. 3 is a flow chart 300 showing a series of example steps for binding metal ions 108 to metal chelating groups 106. It should be understood that the steps shown in flow chart 300 are merely examples, and that in general, a wide variety of different preparative schemes can be used to bind metal atoms or ions 108 to metal chelating groups 106.

In a first optional step 302, the one or more conjugating groups 104 can be protected, for example by addition of a protecting group, prior to introduction of metal ions 108. Examples of suitable protecting groups and synthetic schemes for introducing such groups are disclosed in Elduque et al., “Protected Maleimide Building Blocks for the Decoration of Peptides, Peptoids, and Peptide Nucleic Acids,” Bioconjugate Chem. 24(5): 832-839 (2013), and in Sanchez et al., “Maleimide-Dimethylfuran exo Adducts: Effective Maleimide Protection in the Synthesis of Oligonucleotide Conjugates,” Org. Lett. 13(16): 4364-4367 (2011). The entire contents of each of the foregoing references are incorporated herein by reference.

Next, in step 304, metal chelating groups 106 are exposed to metal ions 108. In general, exposure occurs in solution. In some embodiments, the solution is formed in a polar organic solvent such as DMSO or DMF. In certain embodiments, the solution is formed in distilled water, buffered to a pH of between 1.0 and 7.0. In either type of solution, the un-metallized labeling unit is present at a concentration of between 0.1 mg/mL and 10 mg/mL.

To introduce metal ions 108, an ionic salt of the metal (which is formed from the metal ions 108 and one or more anions such as chloride ions, fluoride ions, iodide ions, bromide ions, nitrate ions, acetate ions, and oxide ions, is introduced into the above solution, in an amount such that a molar ratio of metal ions to metal chelating groups can be approximately 1:1 (e.g., between 0.85:1 and 1:0.85). As an example, wherein the metal ions 108 correspond to La³⁺ ions, suitable ionic salts can include, but are not limited to, LaCl₃, LaF₃, LaI₃, LaBr₃, La(NO₃)₃, La(CH₃CO₂)₃, and La₂O₃.

Exposure typically occurs for between about 5 minutes and about 120 minutes (e.g., between 10 minutes and 90 minutes, between 10 minutes and 60 minutes). During exposure, one or more of the metal ions 108 are chelated to one or more of the chelating groups 106.

Then, in step 306, the now-metallized free labeling units 100 are washed either with additional aliquots of the polar organic solvent or buffered, distilled water, to remove unchelated metal ions. Finally, in optional step 308, the one or more conjugating groups 104 can be de-protected, e.g., by removing protecting groups introduced in step 302. Suitable methods for removing protecting groups are disclosed, for example, in Paris et al., “Exploiting protected maleimides to modify oligonucleotides, peptides and peptide nucleic acids,” Molecules 20(4): 6389-6408 (2015), the entire contents of which are incorporated herein by reference. The procedure ends at step 310, yielding free labeling units 100 in solution.

To preparing a labeling agent composition, the solution of free labeling units 100 can be concentrated. In general, concentrating the free labeling unit solution involves reducing the volume of solvent present in the solution. Typically, even when the volume of solvent is reduced, the anionic counterions added when the metal salt is introduced remain in the composition, and can form a salt with the free labeling units 100.

In some embodiments, the composition including the free labeling units 100 is treated to remove water. Various treatments can be applied to remove water, including dehydration via vacuum centrifuge and/or lyophilization. As a result of such treatments, a percentage by weight of water in the composition can be 10% or less (e.g., 9% or less, 8% or less, 7% or less, 6% or less, 5% or less, 4% or less, 3% or less, 2% or less, 1% or less).

Removal of water to yield compositions with relatively low water content can be an important feature of the compositions described herein. In some embodiments, long term stability of the free labeling units 100 depends upon water content in the composition, because certain conjugating units 104 (such as maleimide-based chemical groups) decompose in the presence of water. By reducing water content in the compositions, long term stability of the compositions can be achieved, allowing storage for longer periods before they are used.

In certain embodiments, the composition can include an organic solvent. The solvent can be introduced during the process of metal chelation (FIG. 3), or afterward. In some embodiments, the solvent can be introduced after water has been removed from the composition. A variety of different polar and non-polar solvents can be used, including, but not limited to, DMF, DMSO, and anhydrous alcohols such as methanol, ethanol, various propanols, and various butanols.

In some embodiments, the composition includes an aqueous solvent, and a pH of the composition is 5.0 or less (e.g., 4.8 or less, 4.6 or less, 4.4 or less, 4.2 or less, 4.0 or less, 3.5 or less, 3.0 or less, 2.5 or less, 2.0 or less, or even less). Maintaining the composition at an acidic pH can help to prevent chelated metal ions from precipitating out of solution, thereby ensuring the free labeling units 100 are not chemically modified prior to their use in sample labeling.

By ensuring that a weight percentage of water in certain compositions is less than 10%, the storage lifetime of the compositions can be significantly increased relative to compositions that include larger weight percentages of water. For example, the storage lifetime of such compositions (e.g., the length of time before which degradation of the composition is measurable) can be two weeks or more (e.g., three weeks or more, four weeks or more, two months or more, four months or more, six months or more, eight months or more, ten months or more, 12 months or more) when the compositions are stored at room temperature. In general, the compositions are typically stored at temperatures of between 30° C. and −80° C. (e.g., −20° C. or less) to reduce the rate at which the compositions degrade.

In certain embodiments, a preservation agent can be added to the composition to lengthen the storage lifetime of the composition. A variety of different preservation agents can be used, including various carbohydrates and polymers, and multiple preservation agents can also be used. Examples of such agents include, but are not limited to, trehalose, maltose, sucrose, polyethylene glycol, and gelatin. Where a preservation agent is added to the composition, a weight percentage of the preservation agent relative to the total mass of preservation agent and free labeling units in the composition can be between 0.5% and 50% (e.g., between 1% and 50%, between 5% and 50%, between 10% and 50%, between 20% and 50%, between 30% and 50%). Where the composition includes a solvent, a concentration of the preservation agent in the solvent can be between 50 mmol/L and 500 mmol/L (e.g., between 50 mmol/L and 250 mmol/L, between 50 mmol/L and 200 mmol/L, between 75 mmol/L and 150 mmol/L, between 100 mmol/L and 200 mmol/L).

In some embodiments, the at least one conjugation group 104 of the free labeling agents 100 in the composition can be protected with a protecting agent, such as a protecting group. The protecting group can either be applied during the metal chelation process (i.e., step 302 in flow chart 300), or after the metal chelation process is complete, during preparation of the free labeling composition. Methods similar to those described above in connection with step 302 can be used to protect the conjugation group(s) 104 during preparation of the labeling composition.

The labeling agent composition that is produced from the above steps isolated and can be packaged for use in subsequent sample labeling processes. To facilitate such processes, the isolated and packaged composition can be included in a reagent kit for sample preparation. In addition to the labeling agent composition, the reagent kit can optionally include a variety of other items for a user of the labeling agent composition, including instructions for storage of the labeling agent composition, instructions for conjugating the free labeling units of the composition to a biological molecule, one or more packaged buffer solutions for use in subsequent sample and/or reagent preparative steps, one or more purification devices for purifying the product of conjugation between the free labeling units of the composition and a biological molecule (e.g., a MWCO column and/or gel permeation chromatography column), and a stabilization solvent to storage of the conjugation product of the free labeling units of the composition and a biological molecule.

### (iv) Conjugated Labeling Reagents

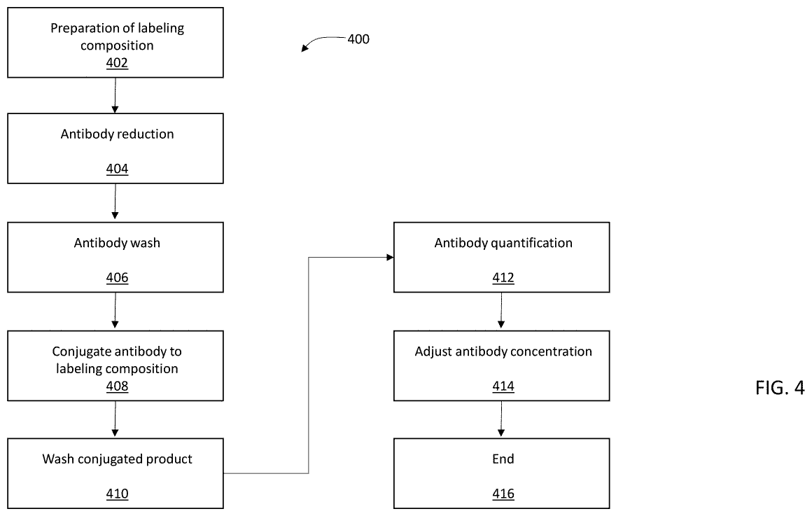

After the labeling agent composition has been produced as described above, the conjugation groups 104 of the free labeling units 100 can be conjugated to biological molecules to produce a conjugated labeling reagent. FIG. 4 is a flow chart 400 that includes example steps for producing a conjugated labeling reagent by covalently bonding conjugating groups 104 to antibodies. It should be understood that in general, a variety of different procedural steps can be used, and that the steps in flow chart 400 represent only examples of suitable steps. Moreover, it should be understood that the free labeling units 100 of the labeling composition can generally be conjugated to biological molecules other than antibodies, such as nucleic acids. Conjugation to an antibody merely serves as an example to further describe preparative steps for sample labeling.

In a first step 402 of flow chart 400, the labeling composition described above is prepared for antibody conjugation. Preparation typically involves optionally equilibrating the labeling composition to room temperature (if necessary) and then performing a short centrifuge operation to collect the free labeling units 100 at the bottom of the centrifuge tube.

Then, in step 404, the antibody is prepared and reduced. Approximately 100 micrograms of antibody is added to a 50 kDa filter device, with a total volume of 400 microliters. If the antibody volume is less than 400 microliters, the difference is volume is made up by adding a sufficient quantity of a buffer solution of 0.01-0.5 M phosphate buffer with 0-10 mM EDTA, pH 7-8. The antibody solution is then centrifuged at 12,000 xg for 10 minutes at room temperature, and the flow-through solution is discarded. Next, 400 microliters of the 0.01-0.5 M phosphate buffer with 0-10 mM EDTA, pH 7-8 buffer solution is added to the antibody and the centrifuging is repeated. Next, 8 microliters of tris(2-carboxyethyl)phosphine (TCEP) are combined with 992 microliters of the 0.01-0.5 M phosphate buffer with 0-10 mM EDTA, pH 7-8 buffer solution to yield a TCEP solution at a concentration of 4 mmol/L. Then, 100 microliters of the 4 mmol/L TCEP solution is added to the concentrated antibody in the 50 kDa filter device, mixing by pipetting the TCEP slowly. Once mixed, the antibody-in-TCEP solution is incubated for 30 minutes at 37° C.

Next, in step 406, the partially reduced antibody from step 404 is washed. A volume of 300 microliters of a tris-buffered salt with 0-10 mM EDTA, pH 7-8 is added to the partially reduced antibody in the 50 kDa filter device, and the mixture is vortexed briefly. Then, the mixture is centrifuged at 12,000 xg for 10 minutes, and the flow-through solution is discarded. Next, 400 microliters of the tris-buffered salt with 0-10 mM EDTA, pH 7-8 is added to the separated antibody, and the mixture is centrifuged again under the same conditions.

In step 408, the partially reduced antibody is conjugated to the labeling composition (and more specifically, to the free labeling units of the labeling composition through the conjugation groups 104). The prepared labeling composition from step 402 is suspended in 200 microliters of the tris-buffered salt with 0-10 mM EDTA, pH 7-8, and transferred into the 50 kDa filter device containing the partially reduced and washed antibody. The labeling composition and antibody are mixed thoroughly by pipetting. The mixture is then incubated at 37° C. for between 60 minutes and 90 minutes to complete the conjugation.

Next, in step 410, the conjugated product is washed. A 200 microliter volume of a 0.01-0.5 M tris-buffered salt solution at a pH of between 7 and 8 is added to the conjugated product in the 50 kDa filter device, and the resulting suspension is centrifuged at 12,000 xg for 10 minutes at room temperature. After discarding the flow-through solution, the washing step is repeated twice more, with fresh 400 microliter aliquots of the 0.01-0.5 M tris-buffered salt solution.

Following washing of the conjugated product, the antibody is quantified in step 412. To perform quantification, 100 microliters of the 0.01-0.5 M tris-buffered salt solution is added to the 50 kDa filter device with thorough mixing by pipetting to yield a conjugated product solution. The concentration of the antibody in the conjugated product is then determined by sampling 2 microliters of the conjugated product solution and measuring that IgG absorbance in the solution sample at 280 nm, with the 0.01-0.5 M tris-buffered salt solution as a background reference. The conjugated product solution is then centrifuged at 12,000 xg for 10 minutes at room temperature to remove the 0.01-0.5 M tris-buffered salt solution.

Next, in step 414, the antibody concentration in the conjugated product is adjusted. A suitable quantity of a 0.01-0.5 M phosphate-buffered salt solution with 0.1-5% bovine serum albumin or gelatin, with 0.01-0.1% sodium azide or Thimerosal, at a pH of between 7 and 8, is added to the 50 kDa filter device, with thorough rinsing of the column walls, to yield a conjugated labeling reagent in which the antibody concentration is approximately 0.5 mg/mL. The filter device is then inverted into a new collection tube and centrifuged at 1,000 xg for 2 minutes at room temperature. Optionally, the conjugated labeling reagent can be filtered by pre-wetting a 0.1 μM filter with 100 microliters of the 0.01-0.5 M phosphate-buffered salt solution, centrifuging at 12,000 xg for 2 minutes and discarding the flow-through solution, and then loading the filter with the conjugated labeling reagent and centrifuging at 12,000 xg for 2 minutes, collecting the flow-through solution as the filtered conjugated labeling reagent.

The conjugated labeling reagent from step 414 can then be stored (e.g., at a temperature of about 4° C.) until sample labeling is to be performed, and the procedure ends at step 416.

### (v) Sample Labeling

In general, the conjugated labeling reagents prepared as described above are compatible with a wide variety of biological samples. Typically, the sample to be imaged is a tissue sample extracted from a human or animal patient. The sample can correspond to a sample of tumor tissue excised during biopsy, or another type of tissue sample retrieved via another invasive surgical or non-invasive procedure.

In some embodiments, the sample corresponds to a formalin-fixed, paraffin-embedded tissue sample. Such samples are commonly prepared during histological workup of biopsied tissue from cancer tumors and other anatomical locations.

In certain embodiments, the sample corresponds to an array of single cells on a substrate. The array can be naturally occurring, and correspond to a regularly occurring, ordered arrangement of cells in a tissue sample. Alternatively, the array of cells can be a product of sample preparation. That is, the sample can be prepared by manual or automated placement of individual cells on substrate (e.g., in a series of wells or depressions formed in a substrate) to form the cell array.

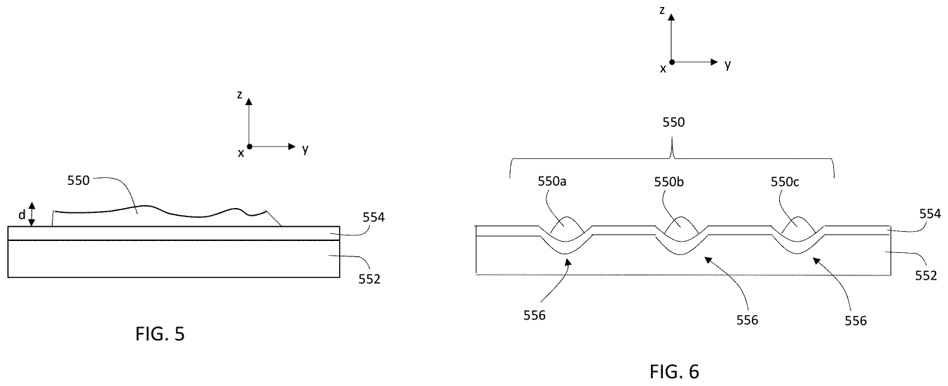

FIG. 5 is a schematic cross-sectional diagram of sample 550 on substrate 552. Positioned between substrate 552 and sample 550 in FIG. 5 is an optional coating 554. When present, coating 554 can be electrically connected to a voltage source via electrodes.

Substrate 552 is typically implemented as a microscope slide or another planar support structure, and can be formed from a variety of materials including various types of glass, plastics, silicon, and metals.

Coating 554, if present, is typically formed of one or more metallic elements, or one or more non-metallic compounds of relatively high conductivity. Examples of metallic elements used to form coating 554 include, but are not limited to, gold, tantalum, titanium, chromium, tin, and indium. In certain embodiments, coating 554 can be implemented as multiple distinct coating layers, each of which can be formed as a separate layer of a metallic element or a separate layer of a relatively high conductivity, non-metallic compound.

As shown in FIG. 5, sample 550 is approximately planar and extends in the x- and/or y-coordinate directions, and has a thickness d measured in the z-coordinate direction. Depending upon the method of preparation of sample 550, the sample can have an approximately constant thickness d across the planar extent of the sample parallel to the x-y coordinate plane. Alternatively, many real samples corresponding to excised tissue have non-constant thicknesses d across the planar extent of the sample parallel to the x-y coordinate plane. In FIG. 5, sample 550—which is shown in cross-section—has a non-constant thickness d measured in the z-coordinate direction.

In some embodiments, substrate 552 can also include one or more additional coating materials to facilitate adhesion of sample 550 to substrate 552. Where no coating 554 is present, the one or more additional coating materials can be applied directly to substrate 550, such that the additional coating materials form a layer positioned between sample 550 and substrate 552. Where coating 554 is present, the one or more additional coating materials can be applied atop coating 554, for example, such that the additional coating materials form a layer positioned between coating 554 and sample 550. Suitable additional coating materials to facilitate adhesion of sample 550 include, but are not limited to, poly-1-lysine.

FIG. 6 shows a schematic cross-sectional diagram of another sample 550 positioned on a substrate 552. Substrate 552 optionally includes one or more conformal coating layers 554 as discussed above. In addition, substrate 552 includes an array of wells 556 corresponding to depressions formed in a surface of substrate 552. Each of the wells 556 contains a portion 550a-550c of sample 550. In general, while substrate 552 includes three wells 556 containing three separate portions 550a-550c of sample 550 in FIG. 6, more generally substrate 552 can include any number of wells 556, and sample 550 can be apportioned among any one or more of the wells 556.

Wells 556 (and the portions of sample 550 distributed among wells 556) can generally arranged in a variety of patterns in substrate 552. For example, wells 556 can form a linear (i.e., one dimensional) array in substrate 552. Alternatively, wells 556 can be distributed along one dimension in the plane of substrate 552, with irregular spacings between some or all of the wells.

As another example, wells 556 can form a two-dimensional array in substrate 552, with regular spacings between adjacent wells in directions parallel to both the x- and y-coordinate directions in the plane of substrate 552. Alternatively, in either or both of the directions parallel to the x- and y-coordinate directions in the plane of substrate 552, at least some of wells 556 can be spaced irregularly.

Where wells 556 form a two-dimensional array in substrate 552, the array can take a variety of forms. In some embodiments, the array of wells 556 can be a square or rectangular array. In certain embodiments, the array can be a hexagonal array, a polar array having radial symmetry, or another type of array having geometrical symmetry in plane of substrate 552.

Each of the portions 550a-550c of sample 550 can include one or more cells. During sample preparation, each portion 550a-550c can be dispensed or positioned in a corresponding well 556 of substrate 552 to form sample 550. For example, each portion 550a-550c of sample 550 can be dispensed into a corresponding well 556 as a suspension of cells in a liquid medium, and the liquid medium subsequently removed (e.g., by washing or heating) to leave the cells in each well 556.

As discussed above, to facilitate various biochemical structural analyses of sample 550 such as protein expression, sample 550 is labeled with multiple mass tags. When sample 550 is exposed to a primary ion beam, the mass tags are ionized and liberated from sample 550. The ionized mass tags correspond to secondary ions emerging from sample 550. Analysis of the secondary ions as a function of the location of incidence of the primary ion beam on sample 550 yields a wealth of information about the biochemical structure of sample 550 at each of the locations of incidence.

To apply the mass tags to sample 550, the sample is exposed to the conjugated labeling reagents prepared as described above. Conjugated labeling reagents that include specific antibodies selectively bind to complementary antigen receptors in sample 550. In practice, solutions of each of the conjugated labeling reagents are prepared, and then sample 550 is labeled by exposing sample 550 to each of the conjugated labeling reagent solutions. Sample 550 is typically exposed to multiple conjugated labeling reagents sequentially and/or in parallel so that sample 550 can be labelled with multiple, distinct mass tags.

For labeling of samples consisting of arrays of cells, cells in suspension can be augmented with surface marker antibodies and incubated at room temperature for approximately 30 minutes. Following incubation, cells can be washed twice with the conjugated labeling reagents to label the cells. Individual aliquots of the labeled cells, diluted in PBS to yield a desired concentration of cells per unit volume (e.g., approximately 10⁷ cells/mL), can then be placed in wells 556 and allowed to adhere for approximately 20 minutes. The adhered cells can then be gently rinsed with PBS, fixed for approximately 5 minutes in PBS with 2% glutaraldehyde, and rinsed twice with deionized water. Samples can then be dehydrated via a graded ethanol series, air dried at room temperature, and stored in a vacuum dessicator for at least 24 hours prior to analysis.

For preparation of intact tissue samples, such as samples obtained from biopsy, tissue samples can be mounted on substrate 552. Following mounting, the samples can be baked at approximately 65° C. for 15 minutes, deparaffinized in xylene (if obtained from FFPE tissue blocks), and rehydrated via a graded ethanol series. The samples are then immersed in epitope retrieval buffer (e.g., 100 mM tris-buffered salt solution with 10 mM EDTA, at pH 9.0) and placed in a pressure cooker (available from Electron Microscopy Sciences, Hatfield, Pa.) for approximately 30 minutes. Subsequently, the samples are rinsed twice with deionized water and once with wash buffer (TBS, 0.1% Tween, pH 7.2). Residual buffer solution can be removed by gently touching the samples with a lint free tissue. The samples are then incubated with blocking buffer for approximately 30 minutes (TBS, 0.1% Tween, 0-3% BSA, 1-10% donkey serum, pH 7.2).

The blocking buffer is then removed and the samples are labeled overnight with the conjugated labeling reagents at 4° C. in a humidified chamber. Following labeling, the samples are rinsed twice in wash buffer, postfixed for approximately 5 minutes (PBS, 2% glutaraldehyde), rinsed in deionized water, and, optionally, stained with Harris hematoxylin for 10 seconds. The samples are then dehydrated via graded ethanol series, air dried at room temperature, and stored in a vacuum dessicator for at least 24 hours prior to analysis.

It should be understood that the above preparative steps are merely provided as examples of methods for sample labeling, and that modifications to the above sequences of steps also yield samples that are suitably labeled with mass tags and prepared for MIBI analysis. In particular, modifications to be above sequences of preparative steps can be undertaken based on the nature of the samples (e.g., the type of tissue to which the samples correspond).

### (vi) Multiplexed Ion Beam Imaging

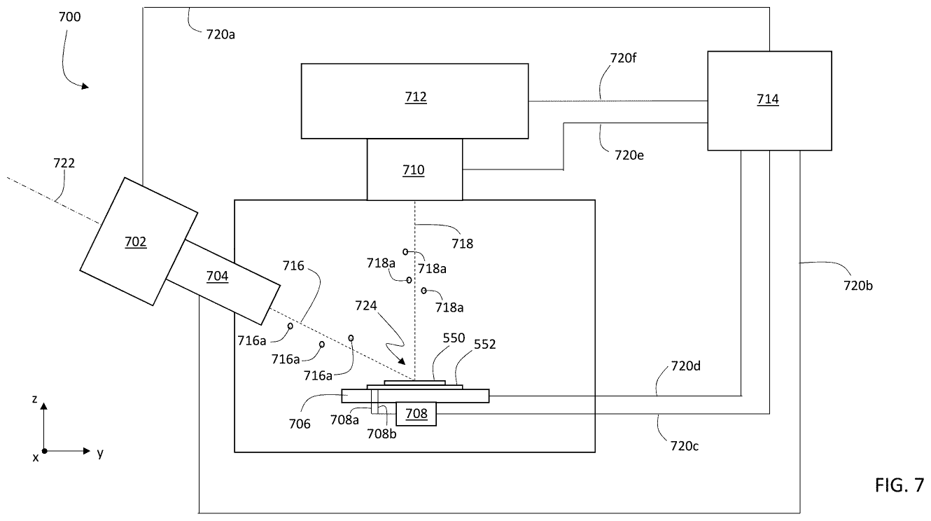

FIG. 7 is a schematic diagram showing an example system 700 for multiplexed ion beam imaging of samples labeled as describe above. System 700 includes an ion beam source 702, ion beam optics 704, a stage 706, a voltage source 708, ion collecting optics 710, and a detection apparatus 712. Each of these components is connected to a controller 714 via signal lines 720a-720f During operation of system 700, controller 714 can adjust operating parameters of each of ion beam source 702, ion beam optics 704, stage 706, voltage source 708, ion collecting optics 710, and detection apparatus 712. Further controller 714 can exchange information with each of the foregoing components of system 700 via signal lines 720a-720f.

During operation, ion beam source 702 generates an ion beam 716 that includes a plurality of primary ions 716a. Ion beam 716 is incident on a sample 550 that is positioned on stage 706. Optionally, in certain embodiments, voltage source 708 applies an electrical potential to substrate 552 that supports sample 550. Primary ions 716a in ion beam 716 interact with sample 550, generating secondary ions 718a as a secondary ion beam 718. Secondary ion beam 718 is collected by ion collecting optics 710 and directed into detection apparatus 712. Detection apparatus 712 measures one or more ion counts corresponding to secondary ions 718a in secondary ion beam 718 and generates electrical signals corresponding the measured ion counts. Controller 714 receives the measured electrical signals from detection apparatus 712 and analyzes the electrical signals to determine information about secondary ions 718a and sample 550.

Controller 714 can adjust a wide variety of different operating parameters of the various components of system 700, and can transmit information (e.g., control signals) and receive information (e.g., electrical signals corresponding to measurements and/or status information) from the components of system 100. For example, in some embodiments, controller 714 can activate ion beam source 702 and can adjust operating parameters of ion beam source 702, such as an ion current of ion beam 716, a beam waist of ion beam 716, and a propagation direction of ion beam 716 relative to central axis 722 of ion beam source 702. In general, controller 714 adjusts the operating parameters of ion beam source 702 by transmitting suitable control signals to ion beam source 702 via signal line 720a. In addition, controller 714 can receive information from ion beam source 702 (including information about the ion current of ion beam 716, the beam waist of ion beam 716, the propagation direction of ion beam 716, and various electrical potentials applied to the components of ion beam source 702) via signal line 720a.

Ion beam optics 704 generally include a variety of elements that use electric fields and/or magnetic fields to control attributes of ion beam 716. In some embodiments, for example, ion beam optics 704 include one or more beam focusing elements that adjust a spot size of ion beam 716 at a location of incidence 724 of ion beam 716 on sample 550. In certain embodiments, ion beam optics 704 include one or more beam deflecting elements that deflect ion beam 716 relative to axis 722, thereby adjusting the location of incidence 724 of ion beam 716 on sample. Ion beam optics 704 can also include a variety of other elements, including one or more apertures, extraction electrodes, beam blocking elements, and other elements that assist in directing ion beam 716 to be incident on sample 550.

Controller 714 can generally adjust the properties of any of the foregoing elements via suitable control signals transmitted via signal line 720b. For example, controller 714 can adjust the focusing properties of one or more beam focusing elements of ion beam optics 704 by adjusting electrical potentials applied to the beam focusing elements via signal line 720b. Similarly, controller 714 can adjust the propagation direction of ion beam 716 (and the location of incidence 724 of ion beam 716 on sample 550) by adjusting electrical potentials applied to the beam deflection elements via signal line 720b. Further, controller 714 can adjust positions of one or more apertures and/or beam blocking elements in ion beam optics 704, and adjust electrical potentials applied to extraction electrodes in ion beam optics 704, via suitable control signals transmitted on signal line 720b. In addition to adjusting properties of ion beam optics 704, controller 714 can receive information from various components of ion beam optics 704, including information about electrical potentials applied to the components of ion beam optics 704 and/or information about positions of the components of ion beam optics 704.

Stage 706 includes a surface for supporting sample 550 (and substrate 552). In general, stage 706 can be translated in each of the x-, y-, and z-coordinate directions. Controller 714 can translate stage 706 in an of the above directions by transmitting control signals on signal line 720d. To effect a translation of the location of incidence 724 of ion beam 716 on sample 550, controller 714 can adjust one or more electrical potentials applied to deflection elements of ion beam optics 704 (e.g., to deflect ion beam 716 relative to axis 722), adjust the position of stage 706 via control signals transmitted on signal line 720d, and/or adjust both deflection elements of ion beam optics 704 and the position of stage 706. In addition, controller 714 receives information about the position of stage 706 transmitted along signal line 720d.

In some embodiments, system 700 includes a voltage source 708 connected to substrate 552 via electrodes 708a and 708b. When activated by controller 714 (via suitable control signals transmitted on signal line 720c), voltage source 708 applies an electrical potential to substrate 552. The applied electrical potential assists in the capture of secondary ion beam 718 from sample 550, as the electrical potential repels secondary ions 718a, causing the secondary ions to leave sample 550 in the direction of ion collecting optics 710.

Secondary ion beam 718 consisting of a plurality of secondary ions 718a is captured by ion collecting optics 710. In general, ion collecting optics 710 can include a variety of electric and magnetic field-generating elements for deflecting and focusing secondary ion beam 718. In addition, ion collecting optics 710 can include one or more apertures, beam blocking elements, and electrodes. As discussed above in connection with ion beam optics 704, controller 714 can adjust electrical potentials applied to each of the components of ion collecting optics 710 via suitable control signals transmitted on signal line 720e. Controller 714 can also adjust the positions of apertures, beam blocking elements, and other movable components of ion collecting optics 710 by transmitting control signals on signal line 720e. In addition, controller 714 can receive information about operating parameters (e.g., voltages, positions) of various components of ion collecting optics 710 on signal line 720e.

Ion collecting optics 710 direct secondary ion beam 718 into detection apparatus 712. Detection apparatus 712 measures ion counts or currents corresponding to the various types of secondary ions 718a in secondary ion beam 718, and generates output signals that contain information about the measured ion counts or currents. Controller 714 can adjust various operating parameters of detection apparatus 712, including maximum and minimum ion count detection thresholds, signal integration times, the range of mass-to-charge (m/z) values over which ion counts are measured, the dynamic range over which ion counts are measured, and electrical potentials applied to various components of detection apparatus 712, by transmitting suitable control signals over signal line 720f.

Controller 714 receives the output signals from detection apparatus that include information about the measured ion counts or currents on signal line 720f. In addition, controller 714 also receives operating parameter information for the various components of detection apparatus 712 via signal line 720f, including values of the various operating parameters discussed above.

Detection apparatus 712 can include a variety of components for measuring ion counts/currents corresponding to secondary ion beam 718. In some embodiments, for example, detection apparatus 712 can correspond to a time-of-flight (TOF) detector. In certain embodiments, detection apparatus 712 can include one or more ion detectors such as Faraday cups, which generate electrical signals when ions are incident on their active surfaces. In some embodiments, detection apparatus 712 can be implemented as a multiplying detector, in which incident ions enter an electron multiplier where they generate a corresponding electron burst. The electron burst can be detected directly as an electrical signal, or can be incident on a converter that generates photons (i.e., an optical signal) in response to the incident electrons. The photons are detected with an optical detector which generates the output electrical signal.

As discussed above, controller 714 is capable of adjusting a wide variety of operating parameters of system 700, receiving and monitoring values of the operating parameters, and receiving electrical signals containing information about secondary ions 718a (and other species) generated from sample 550. Controller 714 analyzes the electrical signals to extract the information about secondary ions 718a and other species. Based on the extracted information, controller 714 can adjust operating parameters of system 700 to improve system performance (e.g., m/z resolution, detection sensitivity) and to improve the accuracy and reproducibility of data (e.g., ion counts) measured by system 700. Controller 714 can also execute display operations to provide system users with images of sample 550 that show distributions of various mass tags within sample 550, and storage operations to store information relating to the distributions in non-volatile storage media.

A variety of different primary ion beams 716 generated by ion source 702 can be used to expose sample 550. In some embodiments, for example, primary ion beam 716 consists of a plurality of oxygen ions (O⁻). For example, ion source 702 can be implemented as an oxygen duoplasmatron source, which generates primary ion beam 716.

To obtain spatially resolved information from sample 550, primary ion beam 716 is translated across sample 550 to multiple different locations of incidence 724. The multiple different locations of incidence form a two-dimensional exposure pattern of primary ion beam 716 in the plane of sample 550 (i.e., in a plane parallel to the x-y plane).

At each location of incidence 724, primary ion beam 716 generates secondary ions 718a that correspond to the antibody-conjugated mass tags bound to sample 550 at that location. The secondary ions 718a—which form secondary ion beam 718—are measured by detection apparatus 712, as ion counts corresponding to different types of secondary ions 718a in secondary ion beam 718. Detection apparatus 712 generates electrical signals corresponding to the measured ion counts. Controller 714 receives the measured electrical signals from detection apparatus 712 and analyzes the electrical signals to determine spatially resolved information about the biochemical structure of sample 550 based on the types and abundance of the secondary ions 718a at each location of incidence 724 on sample 550.

Additional sample preparation steps and features of ion beam imaging systems are described, for example, in U.S. Provisional Patent Application Nos. 62/608,564, 62/621,687, and 62/636,220, the entire contents of each of which are incorporated by reference herein.

(vii) Hardware and Software Implementations

As discussed above, any of the steps and functions described herein can be executed by controller 714. In general, controller 714 can include a single electronic processor, multiple electronic processors, one or more integrated circuits (e.g., application specific integrated circuits), and any combination of the foregoing elements. Software- and/or hardware-based instructions are executed by controller 714 to perform the steps and functions discussed herein. Controller 714 can include a data storage system (including memory and/or storage elements), at least one input device, and at least one output device, such as a display. Each set of software-based instructions, embodied as a software program stored on a tangible, non-transient storage medium (e.g., an optical storage medium such as a CD-ROM or DVD, a magnetic storage medium such as a hard disk, or a persistent solid state storage medium) or device, can be implemented in a high-level procedural or object-oriented programming language, or an assembly or machine language.

### Examples

To demonstrate the effectiveness of the labeling compositions and conjugated labeling reagents disclosed herein, a series of experiments were performed to evaluate the performance of the compositions and reagents during various ion beam imaging operations.

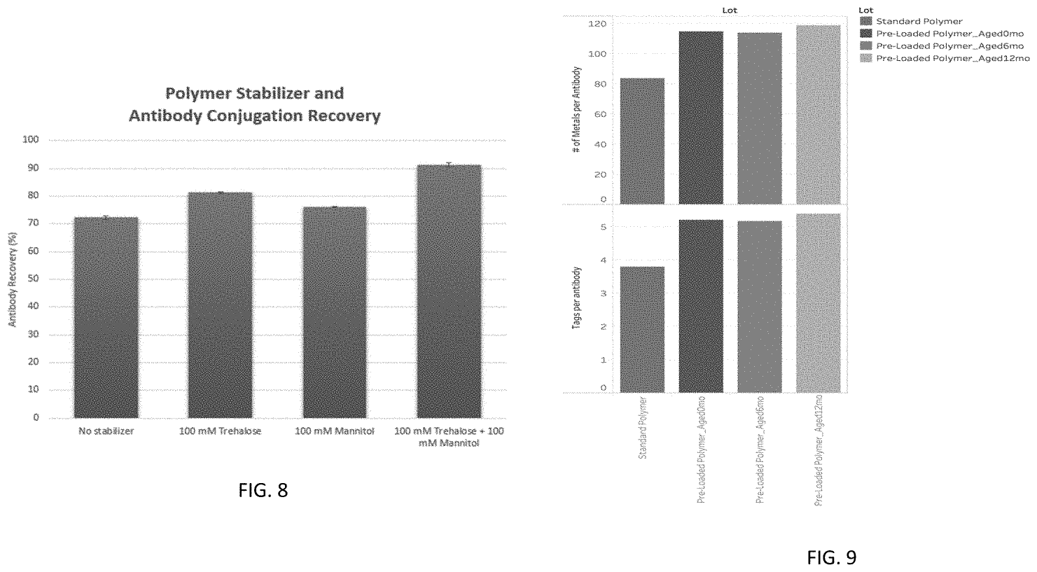

FIG. 8 is a graph showing the amount of antibody recovered following conjugation of the free labeling units to form a conjugated labeling reagent. As shown in the graph, the amount of antibody recovered increases when one or more stabilizers are added to the labeling composition. A combination of trehalose and mannitol added to the labeling composition resulted in a recovery of about 90% of the original antibody following conjugation.

FIG. 9 is a graph showing the results of an inductively-coupled plasma mass spectrometry experiment to quantify the abundance of mass tags per antibody molecule for antibodies conjugated with freshly prepared (in situ) labeling compositions versus labeling compositions that had been prepared previously and stored stabilized at −20° C. (aged 0 months) and at 37° C. for different periods of time to accelerate the aging of the labeling compositions (aged 6 months, 12 months). Antibodies conjugated with previously prepared labeling compositions featured a greater abundance of mass tags, and did not appear to be affected by the the aging process.

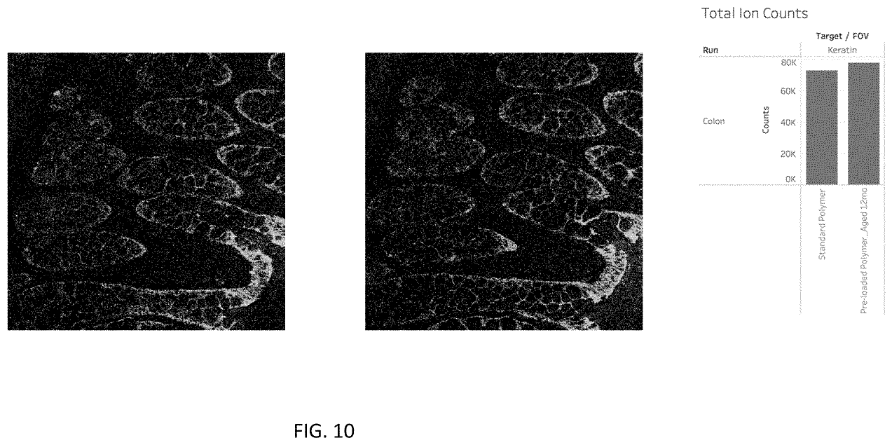

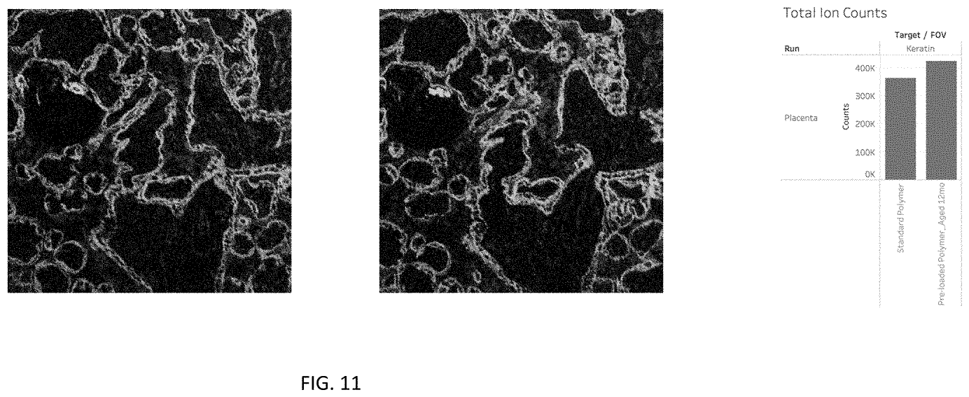

FIGS. 10 and 11 show MIBI images of colon tissue (FIG. 10) and placenta tissue (FIG. 11). Each figure includes a first image (left side) in which the sample tissue is labeled with a keratin-conjugated, metal chelated polymer freshly prepared, and a second image (right side) in which the sample tissue is labeled with a keratin-conjugated labeling composition that had previously been prepared and stored for 12 months. No difference in the measured signal or background secondary ion abundances was observed in either tissue type.

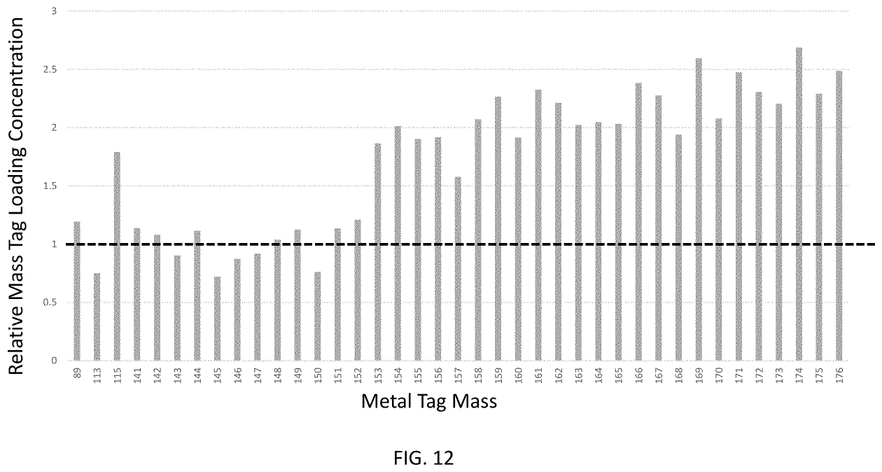

FIG. 12 is a graph showing the results of an inductively-coupled plasma mass spectrometry experiment to quantify the relative loading of various metal tags per polymer molecule in a labeling reagent composition. Thirty nine different metal tag-loaded polymers were prepared and lyophilized according to the methods described herein, and relative concentration levels of metal tags in each polymer were quantified via mass spectrometry. The measured relative concentration levels are shown in the graph as a function of the atomic mass of the conjugated metal tag.

Concentration levels are expressed relative to a value of “1” (as indicated by the dashed line), which corresponds to an experimentally determined concentration level of enriched metal tag incorporation that leads to ideal antibody bioconjugation and metal tag reporter bioassay success. As shown in FIG. 12, all 39 labeling reagents were successfully synthesized at metal tag incorporation levels that are suitable for a wide variety of bioassays.

### OTHER EMBODIMENTS

A number of embodiments have been described. Nevertheless, it will be understood that various modifications may be made without departing from the spirit and scope of the disclosure. Accordingly, other embodiments are within the scope of the following claims.

What is claimed is:

1. A composition, comprising:
a labeling agent comprising a plurality of free labeling units, wherein each labeling unit comprises:
at least one conjugating group for covalent bonding to a biological molecule;
at least one metal chelating group; and
a metal ion chelated by the at least one metal chelating group,
wherein a percentage by weight of water in the composition is 10% or less.

2. The composition of claim 1, wherein each free labeling unit is an oligomeric unit formed from multiple conjugated monomer units.

3. The composition of claim 2, wherein each oligomeric unit comprises at least n conjugated monomer units, wherein n is 3 or more.

4. The composition of claim 2, wherein at least one of the free oligomeric units comprises a first number of conjugated monomer units, and at least one of the free oligomeric units comprises a second number of conjugated monomer units that is different from the first number of conjugated monomer units.

5. The composition of claim 2, wherein at least one of the free oligomeric units comprises one or more first monomer units and one or more second monomer units different from the first monomer units.

6. The composition of claim 5, wherein a ratio of the first monomer units to the second monomer units in the at least one of the free oligomeric units is between 50:1 and 1:50.

7. The composition of claim 5, wherein the first and second monomer units are alternately conjugated in the at least one of the free oligomeric units.

8. The composition of claim 5, wherein at least two of the first monomer units are conjugated to one another in the at least one of the free oligomeric units.

9. The composition of claim 5, wherein at least one of the free oligomeric units comprises one or more first monomer units and one or more third monomer units different from the first and second monomer units.

10. The composition of claim 2, wherein the monomer units comprise one or more amino acids selected from the group consisting of lysine, serine, arginine, glutamine, asparagine, histidine, threonine, tyrosine, cysteine, alanine, isoleucine, leucine, methionine, phenylalanine, valine, proline, and glycine.

11. The composition of claim 2, wherein one or more of the oligomeric units comprise a peptide.

12. The composition of claim 2, wherein the monomer units comprise one or more organic monomers selected from the group consisting of methacrylates and their derivatives, vinyls and their derivatives, ethylenes and their derivatives, propylenes and their derivatives, sulfones and their derivatives, and urethanes and their derivatives.

13. The composition of claim 1, wherein each free labeling unit is a nanoparticle.

14. The composition of claim 1, wherein the at least one conjugating group comprises at least one member selected from the group consisting of: a succinimidyl 4-(N-maleimidomethyl)cycicohexane-1-carboxylate) group, an N-hydroxysuccinimide group, a maleimide group, and an isothiocyanate group.

15. The composition of claim 1, wherein the at least one conjugating group comprises at least one member selected from the group consisting of a moiety that reacts with an amine group of a biological molecule, a moiety that reacts with a sulfhydryl group of a biological molecule, a moiety that reacts with a carboxylic acid group of a biological molecule, a moiety that reacts with acetylene, and a moiety that reacts with an azide group or ion.

16. The composition of claim 1, wherein at least one of the free labeling units comprises multiple conjugating groups.

17. The composition of claim 1, wherein at least one free labeling unit comprises multiple metal chelating groups.

18. The composition of claim 17, wherein the at least one free labeling unit comprises at least one first metal chelating group and at least one second metal chelating group different from the first metal chelating group.

19. The composition of claim 1, wherein the at least one metal chelating group comprises at least one member selected from the group consisting of: a 1,4,7,10-tetraazacyclododecane-1,4,7,10-tetraacetic acid group and conjugates thereof, a diethylenetriaminepentaacetic acid group and conjugates thereof, an ethylenediaminetetraacetic acid group and conjugates thereof, and a 1,4,7-tricarboxymethyl-1,4,7-triazacyclononane group and derivatives thereof.

20. The composition of claim 1, wherein the at least one metal chelating group comprises at least one group having at least two chelating moieties.

21. The composition of claim 1, wherein at least one free labeling unit comprises two or more metal ions.

22. The composition of claim 1, further comprising a plurality of anions such that the composition forms a salt, wherein the plurality of anions comprises at least one member of the group consisting of chloride ions, fluoride ions, iodide ions, bromide ions, nitrate ions, acetate ions, and oxide ions.

23. A method, comprising:
providing a labeling agent comprising a plurality of free labeling units, wherein each labeling unit comprises at least one conjugating group for covalent bonding to biological molecule, at least one metal chelating group, and a metal ion chelated by the at least one metal chelating group, wherein a percentage by weight of water in the labeling agent is 10% or less;
conjugating the labeling agent to a biological molecule to form a conjugated labeling reagent; and
stabilizing the conjugated labeling reagent.

24. The method of claim 23, wherein the stabilized conjugated labeling reagent has a storage lifetime of at least two weeks at room temperature.

25. The method of claim 23, wherein stabilizing the conjugated labeling reagent comprises adjusting a percentage by weight of water in the conjugated labeling reagent to 10% or less.

26. The method of claim 23, wherein stabilizing the conjugated labeling reagent comprises reducing a temperature of the conjugated labeling reagent to −20 degrees Celsius or less, and maintaining the reduced temperature.

27. The method of claim 23, wherein stabilizing the conjugated labeling reagent comprises at least one of adjusting and maintaining a pH of the conjugated labeling reagent to or at a value of 6.5 or less.

28. The method of claim 23, wherein stabilizing the conjugated labeling reagent comprises combining the conjugated labeling reagent with a preservation agent, and wherein the preservation agent comprises at least one member selected from the group consisting of a carbohydrate, a polymer, trehalose, maltose, sucrose, polyethylene glycol, and gelatin.

29. The method of claim 23, wherein stabilizing the conjugated labeling reagent comprises treating the at least one conjugating group with a protecting agent to covalently bind a protecting group to the at least one conjugating group.

30. The method of claim 29, wherein the protecting group comprises at least one member selected from the group consisting of dimethylfuran and its derivatives and isomers such as 2,5-dimethylfuran, methylfuran and its derivatives and isomers such as 2-methylfuran, and chemical groups that form alkoxymethyl ethers such as methoxymethyl ethers and ethoxymethyl ethers.
